SEQUENCE LISTING (3 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 70 aa, Homo sapiens
  MUTAGEN (27)..(27): K27R
GPETLCGAELVDALQFVCGDRGFYFNRPTGYGSSSRRAPQTGIVDECCFRSCDLRRLEMYCAPLRPAKSA

SEQ ID NO: 3 - protein, 70 aa, Homo sapiens
  MUTAGEN (27)..(27): K27R
GPETLCGAELVDALQFVCGDRGFYFNRPTGYGSSSRRAPQTGIVDECCFRSCDLRRLEMYCAPLKPARSA

(19) United States
(12) Patent Application Publication
(10) Pub. No.: US 2011/0152188 A1
(43) Pub. Date: Jun. 23, 2011

(54) PHARMACEUTICAL COMPOSITIONS OF IGF/I PROTEINS

(76) Inventors: Hanns-Christian Mahler, Basel (CH); Astrid Pappenberger, Bettingen (CH)

(21) Appl. No.: 12/969,619

(22) Filed: Dec. 16, 2010

(30) Foreign Application Priority Data
Dec. 23, 2009 (EP) 09180607.5

(51) Int. Cl. A61K 38/30; A61P 25/28; A61P 25/00

(52) U.S. Cl. 514/8.6

(57) ABSTRACT

The present invention relates to a pharmaceutical composition, comprising an Insulin-like growth factor I (IGF-I) protein as active pharmaceutical ingredient (API), a tonicity agent and a buffer. This composition may be administered as injection or infusion and is especially useful for the treatment, prevention and/or delay of progression of neurodegenerative disorders, in particular Alzheimer's Disease (AD), a motor neuron disease (MND), in particular amyotrophic lateral sclerosis (ALS) or Spinal Muscular Atrophy (SMA) or a Muscular Dystrophy (MD), in particular Duchenne Muscular Dystrophy (DMD) or Myotonic Dystrophy (MMD).

### PRIORITY TO RELATED APPLICATION(S)

This application claims the benefit of European Patent Application No. 09180607.5, filed Dec. 23, 2009, which is hereby incorporated by reference in its entirety.

### BACKGROUND OF THE INVENTION

Insulin-like growth factor I (IGF-I) is a circulating anabolic hormone structurally related to insulin. IGF-I was traditionally considered the major mediator of the actions of growth hormone on peripheral tissues. IGF-I consists of 70 amino acids and is also named somatomedin C and is defined by SwissProt No. P01343. Use, activity and production are mentioned in, e.g., le Bouc, Y., et al., FEBS Lett. 196 (1986) 108-112; de Pagter-Holthuizen, P., et al., FEBS Lett. 195 (1986) 179-184; Sandberg Nordqvist, A. C., et al., Brain Res. Mol. Brain. Res. 12 (1992) 275-277; Steenbergh, P. H., et al., Biochem. Biophys. Res. Commun. 175 (1991) 507-514; Tanner, J. M., et al., Acta Endocrinol. (Copenh.) 84 (1977) 681-696; Uthne, K., et al., J. Clin. Endocrinol. Metab. 39 (1974) 548-554; EP 0 123 228; EP 0 128 733; U.S. Pat. No. 5,861,373; U.S. Pat. No. 5,714,460; EP 0 597 033; WO 02/32449; WO 93/02695.

Further information relating to modulation of IGF-I function by IGF-I binding proteins (IGFBP) as well as in-vivo production and occurrence of IGF-I is described in WO 2006/066891 and WO 2009/121759. These references further describe the absorbance, function of IGF-I in the central nervous system (CNS) as well as therapeutic uses. The use of IGF-I for the treatment, prevention and/or delay of progression of neurodegenerative disorders, in particular Alzheimer's Disease (AD), is described in WO 2006/066891. The use of IGF-I for the treatment, prevention and/or delay of progression of neuromuscular disorders is described in WO 2009/121759. It is described therein, that IGF-I is useful in the treatment of motor neuron disease (MND) in particular amyotrophic lateral sclerosis (ALS) or Spinal Muscular Atrophy (SMA) or Muscular Dystrophy (MD), in particular Duchenne Muscular Dystrophy (DMD) or Myotonic Dystrophy (MMD).

WO 2006/066891 discloses PEGylated IGF-I conjugates consisting of an insulin-like growth factor-1 (IGF-I) variant and one or two poly(ethylene glycol) group(s). The described IGF-I variants have an amino acid alteration at up to three amino acid positions 27, 37, 65, 68 of the wild-type IGF-I amino acid sequence so that one or two of said amino acids is/are lysine and amino acid 27 is a polar amino acid but not lysine. Said IGF-I variants are conjugated to PEG via the primary amino group(s) of said lysine(s) and said poly(ethylene glycol) group(s) have an overall molecular weight of 20 to 100 kDa.

PEGylated IGF-I conjugates disclosed in WO 2009/121759 comprise an IGF-I variant that is derived from the wild-type human IGF-I amino acid sequence and carries one or two amino acid alterations at amino acid positions 27, 65 and 68 so that one or two of amino acids at positions 27, 65 and 68 is/are a polar amino acid but not lysine and PEG is attached to at least one lysine residue.

### SUMMARY OF THE INVENTION

The present invention provides a pharmaceutical composition, comprising an Insulin-like growth factor I (IGF-I) protein as active pharmaceutical ingredient (API), a tonicity agent and a buffer. The IGF-I protein may further be conjugated with poly(ethylene glycol) (PEG). This composition may be administered as injection or infusion and is especially useful for the treatment, prevention and/or delay of progression of neurodegenerative disorders, in particular Alzheimer's Disease (AD), a motor neuron disease (MND), in particular amyotrophic lateral sclerosis (ALS) or Spinal Muscular Atrophy (SMA) or a Muscular Dystrophy (MD), in particular Duchenne Muscular Dystrophy (DMD) or Myotonic Dystrophy (MMD).

### DETAILED DESCRIPTION OF THE INVENTION

Unless otherwise indicated the following definitions are set forth to illustrate and define the meaning and scope of the various terms used to describe the invention herein.

The term “IGF-I protein” as used herein refers to an Insulin-like growth factor I as wild-type, any kind of variant as well as to PEGylated IGF-I conjugates thereof.

The term “IGF-I variant” as used herein refers to an IGF-I protein, having an amino acid alteration at amino acid positions 27, 65 and/or 68 of the wild-type IGF-I amino acid sequence (SEQ ID NO: 1). Such IGF-I variants are useful as intermediates for the production of PEGylated IGF-I variants.

IGF-I variants are designated as follows: K27 means that amino acid 27 is lysine, K65 means that amino acid 65 is lysine, K68 means that amino acid 68 is lysine, R27 means that amino acid 27 is arginine, R65 means that amino acid 65 is arginine, R68 means that amino acid 68 is arginine, K27R means that the lysine at amino acid position 27 of SEQ ID NO: 1 is altered to arginine, K65R means that the lysine at amino acid position 65 of SEQ ID NO: 1 is altered to arginine, K68R means that the lysine at amino acid position 28 of SEQ ID NO: 1 is altered to arginine, etc.

A “polar amino acid” as used herein refers to an amino acid selected from the group consisting of cysteine (C), aspartic acid (D), glutamic acid (E), histidine (H), asparagine (N), glutamine (Q), arginine (R), serine (S), and threonine (T). Lysine is also a polar amino acid, but excluded, as lysine is replaced according to the invention. Arginine is preferably used as polar amino acid.

The term “Poly(ethylene glycol)” (or “PEG”) as used herein denotes a residue containing poly(ethylene glycol) as an essential part. Such a PEG can contain further chemical groups which are necessary for binding reactions; which results from the chemical synthesis of the molecule; or which is a spacer for optimal distance of the parts of the molecule from one another. In addition, such a PEG can consist of one or more PEG side-chains which are linked together. Such PEG with more than one PEG chain are called branched. Preferably the PEG have an overall molecular weight of at least 20 kDa, more preferably from about 20 to 100 kDa and especially preferably from 20 to 80 kDa. The PEG is/are preferably branched.

“PEGylated IGF-I variant” as used herein means that an IGF-I variant as defined above is covalently bound to one or two poly(ethylene glycol) groups by amino-reactive coupling to one or two lysines of the IGF-I variant molecule. The PEG group(s) is/are covalently attached at the sites of the IGF-I variant molecule that are the primary ε-amino groups of the lysine side chains. It is further possible that PEGylation occurs in addition on the N-terminal α-amino group. Due to the synthesis method and variant used, PEGylated IGF-I variants can consist of a mixture of IGF-I variants, PEGylated at K65, K68 and/or K27 with or without N-terminal PEGylation, whereby the sites of PEGylation can be different in different molecules or can be substantially homogeneous in regard to the amount of poly(ethylene glycol) side chains per molecule and/or the site of PEGylation in the molecule. Preferably the IGF-I variants are monoPEGylated.

Preferred PEGylated IGF-I variants are PEGylated forms of recombinant human IGF-I variants that have the following amino acid alterations of the wild-type IGF-I amino acid sequence (SEQ ID NO: 1):

### K27R and K65R (SEQ ID NO: 2);

### K27R and K68R (SEQ ID NO: 3).

Special preference is given to the PEGylated form of the recombinant human IGF-I variant with amino acid alterations K27R and K65R (SEQ ID NO: 2) that is mono-PEGylated at K68.

Preference is also given to compositions of a lysine-PEGylated IGF-I variant as described above and an IGF-I variant which is N-terminally PEGylated, wherein said IGF-I variants are identical in terms of the primary amino acid sequence and in that they carry one or two amino acid alterations at amino acid positions 27, 65 and 68 of the wild-type human IGF-I amino acid sequence (SEQ ID NO: 1) so that one or two of amino acids at positions 27, 65 and 68 is/are a polar amino acid but not lysine. Preferably the molecular ratio is 9:1 to 1:9 (ratio means lysine-PEGylated IGF-I variant/N-terminally PEGylated IGF-I variant). Further preferred is a composition wherein the molar ratio is at least 1:1 (at least one part lysine-PEGylated IGF-I variant per one part of N-terminally PEGylated IGF-I variant), preferably at least 6:4 (at least six parts lysine-PEGylated IGF-I variant per four parts of N-terminally PEGylated IGF-I variant). Preferably both the lysine-PEGylated IGF-I variant and the N-terminally PEGylated IGF-I variant are monoPEGylated. Preferably in this composition the variant is identical in both the lysine-PEGylated IGF-I variant and the N-terminally PEGylated IGF-I variant. Preferred PEGylated forms of recombinant human IGF-I variants according to SEQ ID NO. 2 & 3 are obtainable when following the procedure for producing of a lysine-PEGylated IGF-I or a lysine-PEGylated IGF-I variant, said variant comprising one or two amino acid(s) selected from the group consisting of lysine 27, 65 and/or 68 substituted independently by another polar amino acid as described in WO 2008/025528. The process(es) described in WO 2008/025528 allow(s) the preparation of recombinant human IGF-I variants according to SEQ ID NO. 2 & 3, which do not bear N-terminal PEGylation.

It is further preferred, that the PEGylated IGF-I variant is a variant in which up to three (preferably all three) amino acids at the N-terminus are truncated. The respective wild type mutant is named Des(1-3)-IGF-I and lacks the amino acid residues glycine, proline and glutamate from the N-terminus (Kummer, A., et al., Int. J. Exp. Diabesity Res. 4 (2003) 45-57).

The term “PEGylated IGF-I conjugate” as used herein refers to an IGF-I variant covalently bound to one or two PEG as described for PEGylated IGF-I variant.

“MonoPEGylated” as used herein means that IGF-I variant is PEGylated at only one lysine per IGF-I variant molecule, whereby only one PEG group is attached covalently at this site. The pure monoPEGylated IGF-I variant (without N-terminal PEGylation) is at least 80% of the preparation, preferably 90%, and most preferably, monoPEGylated IGF-I variant is 92%, or more, of the preparation, the remainder being e.g. unreacted (non-PEGylated) IGF-I and/or N-terminally PEGylated IGF-I variant. The monoPEGylated IGF-I variant preparations according to the invention are therefore homogeneous enough to display the advantages of a homogeneous preparation, e.g., in a pharmaceutical application. The same applies to the diPEGylated species.

“Substantially homogeneous” as used herein means that the only PEGylated IGF-I variant molecules produced, contained or used are those having one or two PEG group(s) attached. The preparation may contain small amounts of unreacted (i.e., lacking PEG group) protein. As ascertained by peptide mapping and N-terminal sequencing, one example below provides for the preparation which is at least 90% PEGylated IGF-I conjugate and at most 5% unreacted protein. Isolation and purification of such homogeneous preparations of PEGylated IGF-I variant can be performed by usual purification methods, preferably size exclusion chromatography.

As used herein, the term “pharmaceutical composition” (or “composition”) means, e.g., a mixture or solution containing a therapeutically effective amount of an active pharmaceutical ingredient together with pharmaceutically acceptable excipients to be administered to a mammal, e.g., a human in need thereof.

The term “lyophilized composition” (or “lyocomposition”) refers to the composition that is obtained or obtainable by the process of lyophilization of a liquid composition. Typically and preferably it is a solid composition having a water content of less than 5%, preferably of less than 3%.

As used herein the term “lyophilization” refers to the process of freezing a substance and then reducing the concentration of water, by sublimation and/or evaporation to levels which do not support biological or chemical reactions.

As used herein the term “lyophilizate” or “lyophilized form” refers to a solid form of a substance or composition having a water content of less than 5%, obtained by lyophilization.

The terms “reconstituted composition” as used herein in connection with the composition according to the invention denotes a lyophilized composition which is re-dissolved by addition of reconstitution medium. The reconstitution medium comprise but is not limited to water for injection (WFI), bacteriostatic water for injection (BWFI), sodium chloride solutions (e.g. 0.9% (w/v) NaCl), glucose solutions (e.g. 5% glucose), surfactant containing solutions (e.g. 0.01% polysorbate 20), or pH-buffered solution (eg. phosphate-buffered solutions).

An “active pharmaceutical ingredient” (or “API”) is the substance in a pharmaceutical composition that is biologically active

The term “pharmaceutically acceptable excipient” refers to any ingredient having no therapeutic activity and being non-toxic such as disintegrators, binders, fillers, buffers, tonicity agents, stabilizers, antioxidants, surfactants or lubricants used in formulating pharmaceutical products. They are generally safe for administering to humans according to established governmental standards, including those promulgated by the United States Food and Drug Administration.

The term “buffer” as used herein denotes a pharmaceutically acceptable excipient, which stabilizes the pH of a pharmaceutical preparation. Suitable buffers are well known in the art and can be found in the literature. Preferred pharmaceutically acceptable buffers comprise but are not limited to histidine-buffers, citrate-buffers, succinate-buffers, acetate-buffers and phosphate-buffers. Most preferred buffers comprise citrate, L-histidine or mixtures of L-histidine and L-histidine hydrochloride. Other preferred buffer is acetate buffer. Independently from the buffer used, the pH can be adjusted with an acid or a base known in the art, e.g. hydrochloric acid, acetic acid, phosphoric acid, sulfuric acid and citric acid, sodium hydroxide and potassium hydroxide.

The term “tonicity agent” as used herein denotes pharmaceutically acceptable excipient used to modulate the tonicity of a composition. Tonicity in general relates to the osmotic pressure of a solution usually relative to that of human blood serum. The composition can be hypotonic, isotonic or hypertonic. The composition is preferably isotonic. An isotonic composition is liquid or liquid reconstituted from a solid form, e.g. from a lyophilized form and denotes a solution having the same tonicity as some other solution with which it is compared, such as physiologic salt solution and the blood serum. Suitable tonicity agents comprise but are not limited to amino acids and sugars. Preferred tonicity agents are trehalose, sucrose or arginine.

The “tonicity” is a measure of the osmotic pressure of two solutions separated by a semi-permeable membrane. Osmotic pressure is the pressure that must be applied to a solution to prevent the inward flow of water across a semi-permeable membrane. Osmotic pressure and tonicity are influenced only by solutes that cannot cross the membrane, as only these exert an osmotic pressure. Solutes able to freely cross the membrane do not affect tonicity because they will always be in equal concentrations on both sides of the membrane.

The term “amino acid” in context with tonicity agent or stabilizer, denotes a pharmaceutically acceptable organic molecule possessing an amino moiety located at a-position to a carboxylic group. Examples of amino acids include arginine, glycine, ornithine, lysine, histidine, glutamic acid, asparagic acid, isoleucine, leucine, alanine, phenylalanine, tyrosine, tryptophane, methionine, serine, proline. Preferred amino acid in context with tonicity agent or stabilizer is arginine.

The term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra-, penta- and so forth saccharide. In contrast to polysaccharides the monosaccharides and oligosaccharides are water soluble. Examples of oligosaccharides include sucrose, trehalose, lactose, maltose and raffinose. Preferred sugars are sucrose and trehalose, most preferred is trehalose.

The term “surfactant” as used herein denotes a pharmaceutically acceptable excipient which is used to protect protein compositions against mechanical stresses like agitation and shearing. Examples of pharmaceutically acceptable surfactants include poloxamers, polysorbates, polyoxyethylene alkyl ethers (Brij), alkylphenylpolyoxyethylene ethers (Triton-X) or sodium dodecyl sulphate (SDS). Preferred surfactants are polysorbates and poloxamers.

As used herein, the term “polysorbate” refers to oleate esters of sorbitol and its anhydrides, typically copolymerized with ethylene oxide. Preferred polysorbates are Polysorbate 20 (poly(ethylene oxide) (20) sorbitan monolaurate, Tween 20) or Polysorbate 80 (poly(ethylene oxide) (80) sorbitan monolaurate, Tween 80).

The term “poloxamer” as used herein refers to non-ionic triblock copolymers composed of a central hydrophobic chain of poly(propylene oxide) (PPO) flanked by two hydrophilic chains of poly(ethylene oxide) (PEO), each PPO or PEO chain can be of different molecular weights. Poloxamers are also known by the trade name Pluronics. Preferred Poloxamer is Poloxamer 188, a poloxamer wherein the PPO chain has a molecular mass of 1800 g/mol and a PEO content of 80% (w/w).

The term “antioxidant” denotes pharmaceutically acceptable excipients, which prevent oxidation of the active pharmaceutical ingredient. Antioxidants comprise but are not limited to ascorbic acid, glutathione, cysteine, methionine, citric acid, EDTA. Preferred antioxidant is methionine.

As used herein, “neurodegenerative disorder” means a physical condition which has caused or may cause degradation of portion of a subject's nervous system, and include but are not limited to Alzheimer's disease, Parkinson's disease, Huntington's disease, and other similar diseases.

The term “neuromuscular disorders” encompasses diseases that either directly (via intrinsic muscle pathology) or indirectly (via nerve pathology) impair the functioning of muscle. Examples of neuromuscular disorders include but are not limited to Motor Neuron Diseases (MND) like amyotrophic lateral sclerosis ALS (also known as Lou Gehrig's Disease), Spinal Muscular Atrophy (SMA), Spinal Muscular Atrophy Type 1 (SMA1, Werdnig-Hoffmann Disease), Spinal Muscular Atrophy Type 2 (SMA2), Spinal Muscular Atrophy Type 3 (SMA3, Kugelberg-Welander Disease), Spinal Bulbar Muscular Atrophy (SBMA, also known as Kennedy Disease and X-Linked SBMA); or Muscular Dystrophies (MD) like Duchenne Muscular Dystrophy (DMD, also known as Pseudohypertrophic), Becker Muscular Dystrophy (BMD), Emery-Dreifuss Muscular Dystrophy (EDMD), Limb-Girdle Muscular Dystrophy (LGMD), Facioscapulohumeral Muscular Dystrophy (FSH or FSHD, also known as Landouzy-Dejerine), Myotonic Dystrophy (MMD, also known as Steinert Disease), Oculopharyngeal Muscular Dystrophy (OPMD), Distal Muscular Dystrophy (DD, Miyoshi), Congenital Muscular Dystrophy (CMD).

One problem recognized in connection with composition of IGF-I into pharmaceutical products is an undesired aggregation of the polypeptides and therefore a decrease stability. Further, existing compositions of PEG-IGF-I impair a low solubility as well as an increased viscosity, both highly undesirable effects for pharmaceutical compositions for injection or infusion. The such obtained compositions therefore entailed only low concentrations of the active pharmaceutical ingredient.

Therefore, there is a need for pharmaceutical compositions for PEG-IGF-I, which lead to an increased molecule stability, reduced aggregation and provide good solubility and acceptable viscosity even at increased PEG-IGF-I concentrations.

The problem is solved, according to the present invention, by providing a pharmaceutical composition comprising an IGF-I protein, a tonicity agent and a buffer.

Formulating an IGF-I protein in the composition of the invention improves its stability at temperatures above refrigerator temperature (2-8° C.), especially at room temperature (i.e. below 25° C.) and even at higher temperatures, e.g. 40° C. This means that the composition can be stored without cooling for a prolonged period of time, without losing significant amounts of activity and without significant degradation.

Further, the solubility of the IGF-I protein in the composition at physiological pH as well as at refrigerated temperatures is improved considerably.

A further unexpected effect is a reduced overall viscosity of the composition allowing for a considerable increase in the concentration of the IGF-I protein.

In a preferred embodiment, the buffer is either a histidine, citrate, acetate or succinate. Most preferred buffer is histidine or citrate. Other preferred buffer is acetate buffer.

In a preferred embodiment, the buffer has a concentration of 5 to 100 mM.

In a preferred embodiment, the buffer is a histidine buffer of 5 to 100 mM.

In a preferred embodiment, the buffer is a citrate buffer of 5 to 100 mM.

In a preferred embodiment, the pH is between 4.5 and 6.5. Even more preferred is a pH between 5.0 and 6.0.

In a preferred embodiment, the tonicity agent is an amino acid, a sugar or combinations thereof. In an even more preferred embodiment, the tonicity agent is trehalose, sucrose or arginine or combinations thereof, preferably at a concentration of 10 to 1000 mM. Most preferably, the tonicity agent is sucrose, trehalose or arginine. Most preferably, the tonicity agent is at a concentration of 50 to 300 mM.

In a preferred embodiment, the pharmaceutical composition further comprises a surfactant. In an even more preferred embodiment, the surfactant is a polysorbate or a poloxamer or combinations thereof. Preferably, the surfactant is at a concentration of 0.001 to 1% (w/w).

In a further preferred embodiment, the surfactant is polysorbate 20, polysorbate 80 or poloxamer 188, preferably at a concentration of 0.001 to 1% (w/w).

In a further preferred embodiment, the surfactant is polysorbate 20, preferably at a concentration of 0.001 to 0.1% (w/w), more preferably 0.01 to 0.1% (w/w).

In a further preferred embodiment, the surfactant is polysorbate 80, preferably at a concentration of 0.001 to 0.1% (w/w), more preferably 0.01 to 0.1% (w/w).

In a further preferred embodiment, the surfactant is poloxamer 188, preferably at a concentration of 0.001 to 0.1% (w/w), more preferably 0.01 to 0.1% (w/w).

In a preferred embodiment, the pharmaceutical composition further comprises an antioxidant. In an even more preferred embodiment, the antioxidant is methionine. In a preferred embodiment, the antioxidant is at a concentration of 2 to 50 mM.

In a preferred embodiment, the IGF-I protein is an IGF-I variant, that is derived from the wild-type human IGF-I amino acid sequence (SEQ ID NO: 1) and carries one or two amino acid alterations at amino acid positions 27, 65 and 68, so that one or two lysine(s) at positions 27, 65 and 68 is/are arginine.

In a preferred embodiment, the IGF-I protein is a PEGylated IGF-I conjugate. In an even more preferred embodiment, said PEGylated IGF-I conjugate is monoPEGylated at K68 and has the following amino acid alterations of the wild-type human IGF-I amino acid sequence (SEQ ID NO: 1): K27R and K65R (SEQ ID NO: 2). In an evenly preferred embodiment, said PEGylated IGF-I conjugate is mono-PEGylated at K65 and has the following amino acid alterations of the wild-type human IGF-I amino acid sequence (SEQ ID NO: 1): K27R and K68R (SEQ ID NO: 3).

In a preferred embodiment, each PEG of said PEGylated IGF-I conjugate has an overall molecular weight from 20 to 100 kDa.

In a preferred embodiment, each PEG of said PEGylated IGF-I conjugate is a branched PEG.

In a further preferred embodiment, the IGF-I protein is selected from the IGF-I molecules, variants and PEGylated IGF-I conjugates disclosed in WO 2006/066891 or WO 2009/121759 which are incorporated herein by reference.

In a preferred embodiment, the IGF-I protein is present at a concentration of 0.1 to 50 mg/ml.

Even more preferred are embodiments, wherein the IGF-I protein is present at a concentration of 1 to 20 mg/ml.

In a preferred embodiment, the composition comprises a PEGylated IGF-I conjugate which is mono-PEGylated at K68 and has the amino acid alterations K27R and K65R (SEQ ID NO: 2) of the wild-type human IGF-I amino acid sequence at a concentration of 0.1 to 10 mg/ml, arginine at a concentration of 50 to 500 mM, polysorbate 20 at a concentration of 0.001 to 0.01% (w/w) and methionine at a concentration of 5 to 20 mM in a histidine buffer at 1 to 100 mM at a pH of 5.0 to 6.0.

In another preferred embodiment, the composition comprises an IGF-I protein, preferably a PEGylated IGF-I conjugate which is mono-PEGylated at K68 and has the amino acid alterations K27R and K65R (SEQ ID NO: 2) of the wild-type human IGF-I amino acid sequence, at a concentration of 1 to 20 mg/ml in a histidine buffer at 5 to 100 mM at a pH of 5.0 to 6.0, further comprising a combination of a tonicity agent, an optional surfactant and an optional antioxidant selected from the group of:

### Trehalose 50 to 500 mM;

Trehalose 50 to 500 mM and poloxamer 188 0.001 to 0.1% (w/w);
Trehalose 50 to 500 mM and polysorbate 80 or 20 0.001 to 0.1% (w/w);
Trehalose 50 to 500 mM, polysorbate 80 or 20 0.001 to 0.1% (w/w) and methionine 1 to 100 mM;

### Sucrose 50 to 500 mM;

Sucrose 50 to 500 mM and poloxamer 188 0.001 to 0.1% (w/w);
Sucrose 50 to 500 mM and polysorbate 80 or 20 0.001 to 0.1% (w/w);
Sucrose 50 to 500 mM, polysorbate 80 or 20 0.001 to 0.1% (w/w) and methionine 1 to 100 mM;

### Arginine 50 to 500 mM;

Arginine 50 to 500 mM and poloxamer 188 0.001 to 0.1% (w/w);
Arginine 50 to 500 mM and polysorbate 80 or 20 0.001 to 0.1% (w/w); and
Arginine 50 to 500 mM, polysorbate 80 or 20 0.001 to 0.1% (w/w) and methionine 1 to 100 mM.

In another preferred embodiment, the composition comprises an IGF-I protein, preferably a PEGylated IGF-I conjugate which is mono-PEGylated at K68 and has the amino acid alterations K27R and K65R (SEQ ID NO: 2) of the wild-type human IGF-I amino acid sequence, at a concentration of 1 to 20 mg/ml in citrate buffer at 5 to 100 mM at a pH of 5.0 to 6.0, further comprising a combination of a tonicity agent, an optional surfactant and an optional antioxidant selected from the group of:

### Trehalose 50 to 500 mM;

Trehalose 50 to 500 mM and poloxamer 188 (0.001 to 0.1% (w/w));
Trehalose 50 to 500 mM and polysorbate 80 or 20 0.001 to 0.1% (w/w);
Trehalose 50 to 500 mM, polysorbate 80 or 20 0.001 to 0.1% (w/w) and methionine 1 to 100 mM;

### Sucrose 50 to 500 mM;

Sucrose 50 to 500 mM and poloxamer 188 0.001 to 0.1% (w/w);
Sucrose 50 to 500 mM and polysorbate 80 or 20 0.001 to 0.1% (w/w);
Sucrose 50 to 500 mM, polysorbate 80 or 20 0.001 to 0.1% (w/w) and methionine 1 to 100 mM;

### Arginine 50 to 500 mM;

Arginine 50 to 500 mM and poloxamer 188 0.001 to 0.1% (w/w);
Arginine 50 to 500 mM and polysorbate 80 or 20 0.001 to 0.1% (w/w); and
Arginine 50 to 500 mM, polysorbate 80 or 20 0.001 to 0.1% (w/w) and methionine 1 to 100 mM.

In another preferred embodiment, the composition comprises a PEGylated IGF-I conjugate which is mono-PEGylated at K68 and has the amino acid alterations K27R and K65R (SEQ ID NO: 2) of the wild-type human IGF-I amino acid sequence at a concentration of 1 to 20 mg/ml, arginine at a concentration of 50 to 500 mM and poloxamer 188 at a concentration of 0.001 to 0.1% (w/w) in a citrate buffer at 5 to 100 mM at a pH of 5.0 to 6.0.

In another preferred embodiment, the composition comprises a PEGylated IGF-I conjugate which is mono-PEGylated at K68 and has the amino acid alterations K27R and K65R (SEQ ID NO: 2) of the wild-type human IGF-I amino acid sequence at a concentration of 5 to 20 mg/ml, trehalose or sucrose at a concentration of 100 to 200 mM and polysorbate 80 or 20 at a concentration of 0.01 to 0.04% (w/w) in an aqueous buffer prepared from histidine or citrate at 10 to 40 mM at a pH of 5.0 to 6.0.

In a preferred embodiment, the composition is in a liquid form, in a lyophilized form or in a liquid form reconstituted from a lyophilized form.

In a certain embodiment the composition according to the invention is a lyophilized composition. The lyophilized composition according to the invention has the advantage of an improved stability with regard to the formation of particulates and aggregates of higher molecular weight that is usually difficult to be achieved with liquid compositions at the same concentration.

In a preferred embodiment, the composition is prepared in a process, wherein a solution of an IGF-I protein is dialyzed against the buffer intended to be used in the pharmaceutical composition and the desired final protein concentration is adjusted by concentration or dilution.

In a preferred embodiment, the composition is used for the manufacture of a medicament. In a more preferred embodiment, the composition is used for the manufacture of a medicament for the treatment, prevention and/or delay of progression of neurodegenerative disorders, in particular Alzheimer's Disease (AD), a motor neuron disease (MND), in particular amyotrophic lateral sclerosis (ALS) or Spinal Muscular Atrophy (SMA) or a Muscular Dystrophy (MD), in particular Duchenne Muscular Dystrophy (DMD) or Myotonic Dystrophy (MMD).

The compositions of the present invention are especially suitable for the storage of IGF-I proteins in vials, prefilled syringes, ampoules, cartridges, etc.

The compositions of the present invention can be used to stably store IGF-I proteins at different temperatures, including frozen storage, storage under refrigerated conditions or at room temperature for given periods of time.

The composition according to the invention can be administered parenterally, preferably as intravenous (i.v.) or subcutaneous (s.c.) bolus injection, or any other parental administration means such as those known in the pharmaceutical art. The composition can further be administered by infusion as known in the pharmaceutical art.

### EXAMPLES

### Materials and Methods

PEG-IGF-I was produced in analogy to WO 2006/066891.

Sodium acetate buffer was prepared by weighing in the appropriate amount of commercially available acetic acid with subsequent pH adjustment using sodium hydroxide.

Sodium citrate buffer was prepared by weighing in the appropriate amount of commercially available citric acid with subsequent pH adjustment using sodium hydroxide.

Sodium succinate buffer was prepared by weighing in the appropriate amount of commercially available succinic acid with subsequent pH adjustment using sodium hydroxide.

Histidine buffer was prepared by weighing in the appropriate amounts of commercially available L-histidine HCl monohydrate and L-histidine base.

Polysorbate 20 is commercially available. It was diluted by weight to give a highly concentrated stock solution. This stock solution was further diluted into the pharmaceutical compositions.

Polysorbate 80 is commercially available. It was diluted by weight to give a highly concentrated stock solution. This stock solution was further diluted into the pharmaceutical compositions.

Poloxamer 188 is commercially available. It was diluted by weight to give a highly concentrated stock solution. This stock solution was further diluted into the pharmaceutical compositions.

Trehalose dihydrate is commercially available. The appropriate amount of it was weighed in to give a highly concentrated stock solution. This stock solution was further diluted into the pharmaceutical compositions.

Sucrose is commercially available. The appropriate amount of it was weighed in to give a highly concentrated stock solution. This stock solution was further diluted into the pharmaceutical compositions.

L-Arginine HCl is commercially available. The appropriate amount of it was weighed in to give a highly concentrated stock solution. This stock solution was further diluted into the pharmaceutical compositions.

L-Methionine is commercially available. The appropriate amount of it was weighed in to give a highly concentrated stock solution. This stock solution was further diluted into the pharmaceutical compositions.

### Conditions for Stress Tests

Pharmaceutical compositions were subjected to mechanical stress by shaking for 1 week at 2-8° C. and by shaking for 1 week at 25° C. on a horizontal shaker at 200 rpm. Pharmaceutical compositions were subjected to freeze-thaw stress by repeated freezing and thawing at either −20° C. and 2-8° C. or −80° C. and 2-8° C., respectively (5 cycles). Stressed samples were analyzed by a variety of analytical techniques including visual inspection for visible particles, sub visible particles, turbidity, pH, osmolality, protein concentration by UV/VIS spectroscopy, viscosity, reversed phase HPLC(RP-HPLC), size exclusion chromatography (SEC), Karl-Fischer titration (lyophilizates only), NMR spectroscopy, FT-IR spectroscopy and μDSC.

### Stability Testing

Stability of pharmaceutical compositions was tested by putting them on storage at −80° C., −20° C., 2-8° C., 25° C. and 40° C. for up to 8 months. At defined time points, samples were removed from the stability chambers and analyzed by a variety of analytical techniques including visual inspection for visible particles, sub visible particles, turbidity, pH, osmolality, protein concentration by UV/VIS spectroscopy, viscosity, reversed phase HPLC (RP-HPLC), size exclusion chromatography (SEC), Karl-Fischer titration (lyophilizates only), NMR spectroscopy, FT-IR spectroscopy and μDSC.

Size exclusion chromatography (SEC) was performed to detect and quantify the mono-PEGylated IGF-I conjugate (main peak), as well as soluble high molecular weight species (HMW) and low molecular weight hydrolysis products (LMW) in the compositions. HMW species are defined as peaks eluting before the main peak whereas LMW species are eluting after the main peak.

### Example 1

### Preparation of Liquid Compositions

Liquid compositions for intravenous and subcutaneous administration according to the invention were developed as follows:

PEGylated IGF-I conjugates were buffer exchanged and concentrated to an appropriate protein concentration. Subsequently excipients were added as stock solutions. The obtained pharmaceutical compositions were sterile filtered and aseptically filled into sterile glass vials that were closed with rubber stoppers and aluminum caps. All samples were visually inspected and put into the climate chambers in an inverted position.

### Example 2

### Preparation of Lyophilized Compositions

Lyophilized compositions for intravenous and subcutaneous administration according to the invention were developed as follows:

PEGylated IGF-I conjugates were buffer exchanged and concentrated to an appropriate protein concentration. Subsequently excipients were added as stock solutions. The obtained pharmaceutical compositions were sterile filtered and aseptically filled into sterile glass vials. After lyophilization the vials were closed with an aluminum cap and put into the climate chambers.

### Example 3

### Stability of Various Buffer Systems

Compositions were prepared according to example 1 comprising 1 mg/ml of a PEGylated IGF-I conjugate which is mono-PEGylated at K68 and has the amino acid alterations K27R and K65R (SEQ ID NO: 2) of the wild-type human IGF-I amino acid sequence and 20 mM of buffer at various pH values. Stability data after 4 weeks (4 w) storage at 40° C. is presented in table 1. An increase of high molecular weight species (HMW) during storage compared to the initial value is indicative for aggregation of PEGylated IGF-I conjugates, whereas an increase of low molecular weight species (LMW) during storage compared to initial value is indicative for a degradation of PEGylated IGF-I conjugates, e.g. by cleavage of branched PEG side chains.

TABLE 1
Stability of various compositions at an API concentration of 1 mg/ml in
dependency of buffer as determined by SEC.

| | Buffer | | | | | | |
|---|---|---|---|---|---|---|---|
| | | | | | | Histidine/ | |
| | Na Acetate | | | Na Citrate | | Histidine HCl | |
| pH | 4.5 | 5.0 | 5.5 | 5.0 | 5.5 | 5.5 | 6.0 |
| HMW [%] | 0.7 | 0.7 | 0.7 | 0.7 | 0.7 | 0.7 | 0.8 |
| initial | | | | | | | |
| LMW [%] | 0.0 | 0.0 | 0.0 | 0.0 | 0.0 | 0.0 | 0.0 |
| initial | | | | | | | |
| HMW [%] | 5.9 | 2.3 | 1.5 | 1.0 | 1.0 | 1.0 | 1.1 |
| 4 w @ 40° C. | | | | | | | |
| LMW [%] | 10.1 | 3.8 | 1.2 | 0.0 | 0.0 | 0.0 | 0.0 |
| 4 w @ 40° C. | | | | | | | |

Further compositions were prepared according to example 1 comprising 8 mg/ml of a PEGylated IGF-I conjugate which is mono-PEGylated at K68 and has the amino acid alterations K27R and K65R (SEQ ID NO: 2) of the wild-type human IGF-I amino acid sequence and 20 mM of buffer at various pH values. Stability data after 7 weeks (7 w) storage at 40° C. is presented in table 2. An increase of high molecular weight species (HMW) during storage compared to the initial value is indicative for aggregation of PEGylated IGF-I conjugates, whereas an increase of low molecular weight species (LMW) during storage compared to initial value is indicative for a degradation of PEGylated IGF-I conjugates, e.g. by cleavage of branched PEG side chains.

TABLE 2
Stability of various compositions at an API concentration of 8 mg/ml in
dependency of buffer as determined by SEC.

| | Buffer | | | | | | | | | |
|---|---|---|---|---|---|---|---|---|---|---|
| | | | | | | | Histidine/ | | | Na |
| | Na Citrate | | | Na Succinate | | | Histidine HCl | | | Acetate |
| pH | 5.0 | 5.5 | 6.0 | 5.0 | 5.5 | 6.0 | 5.5 | 6.0 | 6.5 | 5.5 |
| HMW | 1.4 | 1.4 | 1.3 | 1.5 | 1.4 | 1.4 | 1.2 | 1.3 | 1.5 | 1.4 |
| [%] | | | | | | | | | | |
| initial | | | | | | | | | | |
| LMW | 0.1 | 0.1 | 0.1 | 0.1 | 0.1 | 0.1 | 0.1 | 0.1 | 0.1 | 0.1 |
| [%] | | | | | | | | | | |
| initial | | | | | | | | | | |
| HMW | 3.4 | 3.9 | 9.5 | 24.6 | 18.5 | 6.7 | 4.2 | 31.4 | 17.3 | 4.6 |
| [%] | | | | | | | | | | |
| 7 w @ | | | | | | | | | | |
| 40° C. | | | | | | | | | | |
| LMW | 0.1 | 0.1 | 0.1 | 22.1 | 21.2 | 3.0 | 0.1 | 33.0 | 1.9 | 0.9 |
| [%] | | | | | | | | | | |
| 7 w @ | | | | | | | | | | |
| 40° C. | | | | | | | | | | |

### Example 4

### Effect of Surfactant on Stability

Compositions were prepared according to example 1 comprising 6 mg/ml of a PEGylated IGF-I conjugate which is mono-PEGylated at K68 and has the amino acid alterations K27R and K65R (SEQ ID NO: 2) of the wild-type human IGF-I amino acid sequence, either 20 mM of Histidine/Histidine HCl buffer or 20 mM Na Citrate buffer at pH 5.5 and optionally a surfactant selected from Polysorbate 20, Polysorbate 80 and Poloxamer 188. Results of visual inspection after stress testing and stability data after 26 weeks (26 w) storage at 40° C. are presented in tables 3 & 4. An increase of high molecular weight species (HMW) during storage compared to the initial value is indicative for aggregation of PEGylated IGF-I conjugates, whereas an increase of low molecular weight species (LMW) during storage compared to initial value is indicative for a degradation of PEGylated IGF-I conjugates, e.g. by cleavage of branched PEG side chains.

TABLE 3
Results of visual inspection and stability of various compositions
at an API concentration of 6 mg/ml in Histidine/Histidine HCl buffer
at pH 5.5 in dependency of surfactant upon stress testing
(repeated freeze-thaw at −80° C. and 2-8° C.,
shaking at 2-8° C. and shaking at 25° C.).

| | Surfactant | | | | | | | | | |
|---|---|---|---|---|---|---|---|---|---|---|
| | none | Polysorbate 20 | | | Polysorbate 80 | | | Poloxamer 188 | | |
| Conc. [% w/w] | — | 0.01 | 0.03 | 0.05 | 0.01 | 0.03 | 0.05 | 0.01 | 0.03 | 0.05 |
| Repeated | + | − | − | − | + | + | + | + | + | + |
| freeze-thaw | | | | | | | | | | |
| stress | | | | | | | | | | |
| Shaking | + | + | + | + | + | + | + | + | + | + |
| @ 2-8° C. | | | | | | | | | | |
| Shaking | + | + | + | + | + | − | − | + | + | + |
| @ 25° C. | | | | | | | | | | |
| HMW [%] | 3.3 | 3.3 | 3.3 | 3.3 | 3.3 | 3.3 | 3.3 | 3.3 | 3.3 | 3.3 |
| initial | | | | | | | | | | |
| LMW [%] | 0.2 | 0.2 | 0.2 | 0.2 | 0.1 | 0.1 | 0.1 | 0.2 | 0.2 | 0.2 |
| initial | | | | | | | | | | |
| HMW [%] | 36.4 | 37.4 | 38.9 | 42.1 | 38.5 | 37.9 | 38.6 | 39.5 | 35.9 | 42.8 |
| 26 w @ 40° C. | | | | | | | | | | |
| LMW [%] | 20.4 | 20.4 | 22.7 | 22.4 | 21.8 | 20.9 | 21.8 | 22.1 | 21.4 | 21.9 |
| 26 w @ 40° C. | | | | | | | | | | |

“−”: visible particles detected.
“+”: no visible particles detected.

TABLE 4
Results of visual inspection and stability of various compositions
at an API concentration of 6 mg/ml in Na Citrate buffer
at pH 5.5 in dependency of surfactant upon stress testing
(repeated freeze-thaw at −80° C. and 2-8° C.,
shaking at 2-8° C. and shaking at 25° C.).

| | Surfactant | | | | | | | | | |
|---|---|---|---|---|---|---|---|---|---|---|
| | none | Polysorbate 20 | | | Polysorbate 80 | | | Poloxamer 188 | | |
| Conc. [% w/w] | — | 0.01 | 0.03 | 0.05 | 0.01 | 0.03 | 0.05 | 0.01 | 0.03 | 0.05 |
| Repeated | + | − | − | − | + | + | + | + | + | + |
| freeze-thaw | | | | | | | | | | |
| stress | | | | | | | | | | |
| Shaking | + | + | + | + | + | + | + | + | + | + |
| @ 2-8° C. | | | | | | | | | | |
| Shaking | + | + | − | + | + | + | + | + | + | + |
| @ 25° C. | | | | | | | | | | |
| HMW [%] | 3.4 | 3.5 | 3.5 | 3.4 | 3.5 | 3.4 | 3.5 | 3.5 | 3.4 | 3.5 |
| initial | | | | | | | | | | |
| LMW [%] | 0.1 | 0.1 | 0.1 | 0.1 | 0.1 | 0.1 | 0.1 | 0.1 | 0.1 | 0.1 |
| initial | | | | | | | | | | |
| HMW [%] | 19.4 | 20.3 | 20.3 | 21.0 | 21.6 | 20.5 | 20.3 | 20.8 | 21.6 | 20.6 |
| 26 w @ 40° C. | | | | | | | | | | |
| LMW [%] | 0.1 | 0.1 | 0.1 | 0.1 | 0.1 | 0.1 | 0.1 | 0.1 | 0.1 | 0.1 |
| 26 w @ 40° C. | | | | | | | | | | |

“−”: visible particles detected.
“+”: no visible particles detected.

### Example 5

### Effect of Tonicity Agent and Antioxidant on Stability and Viscosity

Compositions were prepared according to example 1 comprising 6 mg/ml of a PEGylated IGF-I conjugate which is mono-PEGylated at K68 and has the amino acid alterations K27R and K65R (SEQ ID NO: 2) of the wild-type human IGF-I amino acid sequence, either 20 mM of Histidine/Histidine HCl buffer or 20 mM Na Citrate buffer at pH 5.5, optionally a surfactant selected from Polysorbate 80 and Poloxamer 188 at a concentration of 0.01% w/w, a tonicity agent selected from Trehalose (Tre, 220 mM), Sucrose (Suc, 200 mM) and Arginine HCl (Arg, 142 mM) and optionally Methionine (Met, 10 mM) as antioxidant. Viscosity data of the initial analysis, stability data after 12 weeks (12 w) storage at 40° C. and results of visual inspection after 6 months (6 m) at 25° C. are presented in tables 5 & 6. An increase of high molecular weight species (HMW) during storage compared to the initial value is indicative for aggregation of PEGylated IGF-I conjugates.

TABLE 5
Viscosity at initial analysis, stability after 12 weeks at 40° C. and results of visual inspection after
6 months at 25° C. of various compositions at an API concentration of 6 mg/ml in Histidine/
Histidine HCl buffer at pH 5.5 in dependency of surfactant, tonicity agent and antioxidant.

| | Surfactant | | | | | | | | | | | |
|---|---|---|---|---|---|---|---|---|---|---|---|---|
| | none | | | Polysorbate 80 | | | | | | Poloxamer 188 | | |
| Tonicity agent | Tre | Suc | Arg | Tre | | Suc | | Arg | | Tre | Suc | Arg |
| Antioxidant | — | — | — | — | Met | — | Met | — | Met | — | — | — |
| Viscosity | >10 | >10 | <10 | >10 | >10 | >10 | >10 | <10 | <10 | >10 | >10 | <10 |
| [mPa · s] | | | | | | | | | | | | |
| HMW [%] | 3.5 | 3.5 | 3.5 | 3.5 | 3.5 | 3.5 | 3.5 | 3.5 | 3.5 | 3.6 | 3.5 | 3.5 |
| initial | | | | | | | | | | | | |
| HMW [%] | 8.6 | 7.9 | 6.8 | 8.3 | 10.0 | 7.7 | 10.2 | 6.1 | 7.8 | 8.8 | 7.9 | 7.0 |
| 12 w @ 40° C. | | | | | | | | | | | | |
| Visual | + | + | + | − | − | − | − | − | − | + | + | + |
| inspection | | | | | | | | | | | | |

“−”: visible particles detected.
“+”: no visible particles detected.

TABLE 6
Viscosity at initial analysis, stability after 12 weeks at 40° C. and results of visual
inspection after 6 months at 25° C. of various compositions at an API concentration of 6 mg/ml in
Na citrate buffer at pH 5.5 in dependency of surfactant, tonicity agent and antioxidant.

| | Surfactant | | | | | | | | | | | |
|---|---|---|---|---|---|---|---|---|---|---|---|---|
| | none | | | Polysorbate 80 | | | | | | Poloxamer 188 | | |
| Tonicity agent | Tre | Suc | Arg | Tre | | Suc | | Arg | | Tre | Suc | Arg |
| Antioxidant | — | — | — | — | Met | — | Met | — | Met | — | — | — |
| Viscosity | >10 | >10 | <10 | >10 | >10 | >10 | >10 | <10 | <10 | >10 | >10 | <10 |
| [mPa · s] | | | | | | | | | | | | |
| HMW [%] | 3.2 | 3.3 | 3.2 | 3.2 | 3.2 | 3.2 | 3.2 | 3.2 | 3.2 | 3.2 | 3.2 | 3.2 |
| initial | | | | | | | | | | | | |
| HMW [%] | 8.0 | 7.5 | 5.6 | 8.0 | 8.5 | 7.2 | 7.5 | 5.6 | 6.4 | 8.0 | 7.6 | 5.6 |
| 12 w @ 40° C. | | | | | | | | | | | | |
| Visual | + | + | + | − | − | − | − | − | − | + | + | + |
| inspection | | | | | | | | | | | | |

“−”: visible particles detected.
“+”: no visible particles detected.

### Example 6

### Effect of Buffer, Surfactant and Tonicity Agent on Stability and Viscosity of Lyocompositions

Compositions were prepared according to example 2 comprising 6 mg/ml of a PEGylated IGF-I conjugate which is mono-PEGylated at K68 and has the amino acid alterations K27R and K65R (SEQ ID NO: 2) of the wild-type human IGF-I amino acid sequence, either 20 mM of Histidine/Histidine HCl buffer or 20 mM Na Citrate buffer at pH 5.5, Polysorbate 80 (0.01% w/w) as surfactant and Sucrose (220 mM) as tonicity agent. Viscosity data and stability data after 12 weeks (12 w) storage at 40° C. are presented in table 7. An increase of high molecular weight species (HMW) during storage compared to the initial value is indicative for aggregation of PEGylated IGF-I conjugates.

TABLE 7
Stability and viscosity of various lyocompositions at an API concentration
of 6 mg/ml in dependency of buffer, surfactant and tonicity agent.

Buffer

| | |
|---|---|
| Histidine/Histidine HCl | Na Citrate |

Surfactant

| | |
|---|---|
| Polysorbate 80 | Polysorbate 80 |

Tonicity agent

| | Sucrose | Sucrose |
|---|---|---|
| Viscosity [mPa · s] | >10 | >10 |
| HMW [%] | 3.5 | 3.3 |
| initial | | |
| HMW [%] | 3.7 | 3.8 |
| 12 w @ 40° C. | | |

### Example 7

### Effect of Buffer, Surfactant and Tonicity Agent on Stability of Highly Concentrated Lyocompositions

Compositions were prepared according to example 2 comprising 12 mg/ml of a PEGylated IGF-I conjugate (after reconstitution of a 6 mg/ml PEGylated IGF-I conjugate lyophilizate) which is mono-PEGylated at K68 and has the amino acid alterations K27R and K65R (SEQ ID NO: 2) of the wild-type human IGF-I amino acid sequence, either 20 mM of Histidine/Histidine HCl buffer or 20 mM Na Citrate buffer at pH 5.5, Polysorbate 80 (0.02% w/w) as surfactant and Sucrose (130 mM) or Trehalose (130 mM) as tonicity agent. Stability data after 9 weeks (9 w) storage at 40° C. is presented in table 8. An increase of high molecular weight species (HMW) during storage compared to the initial value is indicative for aggregation of PEGylated IGF-I conjugates.

TABLE 8
Stability of various lyocompositions at an API concentration of
12 mg/ml in dependency of buffer, surfactant and tonicity agent.

| Buffer | |
|---|---|
| Histidine/Histidine | |
| HCl | Na Citrate |
| Surfactant | |
| Polysorbate 80 | Polysorbate 80 |

Tonicity agent

| | Trehalose | Sucrose | Trehalose | Sucrose |
|---|---|---|---|---|
| HMW [%] | 3.5 | 3.6 | 3.2 | 3.2 |
| initial | | | | |
| HMW [%] | 3.7 | 3.7 | 4.0 | 3.9 |
| 9 w @ 40° C. | | | | |

1. A pharmaceutical composition, comprising an IGF-I protein, a tonicity agent and a buffer.

2. The pharmaceutical composition of claim 1, wherein the buffer is a histidine, citrate, acetate or succinate buffer and wherein the pH of the composition is between 4.5 and 6.5.

3. The pharmaceutical composition of claim 2, wherein the pH is between 5.0 and 6.0.

4. The pharmaceutical composition of claim 2, wherein the buffer is a citrate buffer present in a concentration of 5 to 100 mM.

5. The pharmaceutical composition of claim 2, wherein the buffer is a histidine buffer present in a concentration of 5 to 100 mM.

6. The pharmaceutical composition of claim 1, wherein the tonicity agent is an amino acid, a sugar or combinations thereof.

7. The pharmaceutical composition of claim 6, wherein the tonicity agent is trehalose, sucrose or arginine or combinations thereof present at a concentration of 10 to 1000 mM.

8. The pharmaceutical composition of claim 6, wherein the tonicity agent is arginine present at a concentration of 50 to 300 mM

9. The pharmaceutical composition of claim 1, further comprising a surfactant.

10. The pharmaceutical composition of claim 9, wherein the surfactant is a polysorbate or a poloxamer or combinations thereof.

11. The pharmaceutical composition of claim 10, wherein the surfactant is polysorbate 20, polysorbate 80 or poloxamer 188 present at a concentration of 0.001 to 1% (w/w).

12. The pharmaceutical composition of claim 1, further comprising an antioxidant.

13. The pharmaceutical composition of claim 12, wherein the antioxidant is methionine present at a concentration of 1 to 50 mM.

14. The pharmaceutical composition of claim 1, wherein the IGF-I protein is an IGF-I variant, derived from the wild-type human IGF-I amino acid sequence (SEQ ID NO: 1) which carries one or two amino acid alterations at amino acid positions 27, 65 and 68, so that one or two lysine(s) at positions 27, 65 and 68 is/are arginine.

15. The pharmaceutical composition of claim 1, wherein the IGF-I protein is a PEGylated IGF-I conjugate.

16. The pharmaceutical composition of claim 15, wherein said PEGylated IGF-I conjugate is mono-PEGylated at K68 and has the amino acid alterations K27R and K65R (SEQ ID NO: 2) of the wild-type human IGF-I amino acid sequence.

17. The pharmaceutical composition of claim 15, wherein said PEGylated IGF-I conjugate is mono-PEGylated at K65 and has the amino acid alterations K27R and K68R (SEQ ID NO: 3) of the wild-type human IGF-I amino acid sequence.

18. The pharmaceutical composition of claim 15, wherein each PEG of said PEGylated IGF-I conjugate has an overall molecular weight from 20 to 100 kDa.

19. The pharmaceutical composition of claim 15, wherein each PEG of said PEGylated IGF-I conjugate is a branched PEG.

20. The pharmaceutical composition of claim 1, wherein the IGF-I protein is present at a concentration of 0.1 to 50 mg/ml.

21. The pharmaceutical composition of claim 20, wherein the IGF-I protein is present at a concentration of 1 to 20 mg/ml.

22. The pharmaceutical composition of claim 1, having a viscosity below 40 mPa·s.

23. The pharmaceutical composition of claim 1, wherein the IGF-I protein comprises PEGylated IGF-I conjugate which is mono-PEGylated at K68 and has the amino acid alterations K27R and K65R (SEQ ID NO: 2) of the wild-type human IGF-I amino acid sequence and is present at a concentration of 0.1 to 10 mg/ml, the tonicity agent comprises arginine at a concentration of 50 to 500 mM, polysorbate 20 at a concentration of 0.001 to 0.01% (w/w) and methionine at a concentration of 1 to 100 mM and the buffer comprises histidine at 5 to 100 mM and wherein the pH of the composition is 5.0 to 6.0.

24. The pharmaceutical composition of claim 1, wherein the IGF-I protein comprises a PEGylated IGF-I conjugate which is mono-PEGylated at K68 and has the amino acid alterations K27R and K65R (SEQ ID NO: 2) of the wild-type human IGF-I amino acid sequence and is present at a concentration of 1 to 20 mg/ml, the buffer comprises histidine at 5 to 100 mM and the composition has a pH of 5.0 to 6.0, further comprising a combination of a tonicity agent, an optional surfactant and an optional antioxidant selected from the group of:
50 to 500 mM Trehalose;
50 to 500 mM Trehalose and 0.001 to 0.1% (w/w) poloxamer 188;
50 to 500 mM Trehalose and 0.001 to 0.1% (w/w) polysorbate 80 or 20;
50 to 500 mM Trehalose, 0.001 to 0.1% (w/w) polysorbate 80 or 20 and 1 to 100 mM methionine;
50 to 500 mM Sucrose;
50 to 500 mM Sucrose and 0.001 to 0.1% (w/w) poloxamer 188;
50 to 500 mM Sucrose and 0.001 to 0.1% (w/w) polysorbate 80 or 20;
50 to 500 mM Sucrose, 0.001 to 0.1% (w/w) polysorbate 80 or 20 and 1 to 100 mM methionine;
50 to 500 mM Arginine;
50 to 500 mM Arginine and 0.001 to 0.1% (w/w) poloxamer 188;
50 to 500 mM Arginine and 0.001 to 0.1% (w/w) polysorbate 80 or 20; and
50 to 500 mM Arginine, 0.001 to 0.1% (w/w) polysorbate 80 or 20 and 1 to 100 mM methionine.

25. The pharmaceutical composition of claim 1, wherein the IGF_I protein comprises a PEGylated IGF-I conjugate which is mono-PEGylated at K68 and has the amino acid alterations K27R and K65R (SEQ ID NO: 2) of the wild-type human IGF-I amino acid sequence and is present at a concentration of 1 to 20 mg/ml in a citrate buffer at 5 to 100 mM wherein the composition has a pH of 5.0 to 6.0, further comprising a combination of a tonicity agent, an optional surfactant and an optional antioxidant selected from the group of:
50 to 500 mM Trehalose;
50 to 500 mM Trehalose and 0.001 to 0.1% (w/w) poloxamer 188;
50 to 500 mM Trehalose and 0.001 to 0.1% (w/w) polysorbate 80 or 20;
50 to 500 mM Trehalose, 0.001 to 0.1% (w/w) polysorbate 80 or 20 and 1 to 100 mM methionine;
50 to 500 mM Sucrose;
50 to 500 mM Sucrose and 0.001 to 0.1% (w/w) poloxamer 188;
50 to 500 mM Sucrose and 0.001 to 0.1% (w/w) polysorbate 80 or 20;
50 to 500 mM Sucrose 0.001 to 0.1% (w/w) polysorbate 80 or 20 and 1 to 100 mM methionine;
50 to 500 mM Arginine;
50 to 500 mM Arginine and 0.001 to 0.1% (w/w) poloxamer 188;
50 to 500 mM Arginine and 0.001 to 0.1% (w/w) polysorbate 80 or 20; and
50 to 500 mM Arginine, 0.001 to 0.1% (w/w) polysorbate 80 or 20 and 1 to 100 mM methionine.

26. The pharmaceutical composition of claim 1, wherein the IGF_I protein comprises PEGylated IGF-I conjugate which is mono-PEGylated at K68 and has the amino acid alterations K27R and K65R (SEQ ID NO: 2) of the wild-type human IGF-I amino acid sequence and is present at a concentration of 1 to 20 mg/ml, the tonicity agent comprises arginine at a concentration of 50 to 500 mM and poloxamer 188 at a concentration of 0.001 to 0.1% (w/w) and wherein the buffer comprises citrate at 5 to 100 mM and wherein the composition has a pH of 5.0 to 6.0.

27. The pharmaceutical composition of claim 1, wherein the IGF-I protein comprises a PEGylated IGF-I conjugate which is mono-PEGylated at K68 and has the amino acid alterations K27R and K65R (SEQ ID NO: 2) of the wild-type human IGF-I amino acid sequence and is present at a concentration of 5 to 20 mg/ml, the tonicity agent comprises trehalose or sucrose at a concentration of 100 to 200 mM and polysorbate 80 or 20 at a concentration of 0.01 to 0.04% (w/w) and the buffer comprises histidine or citrate at 10 to 40 mM and wherein the composition has a pH of 5.0 to 6.0.

28. The pharmaceutical composition of claim 1, which is in a liquid form, is a lyophilized composition or is a reconstituted composition.

29. The composition of claim 1 comprising a parenteral, intravenous (i.v.) or subcutaneous (s.c.) bolus injection form.
